(19) United States
(12) Patent Application Publication
(10) Pub. No.: US 2026/0263340 A1
(43) Pub. Date: Sep. 10, 2026

(54) Cosmetic Products and Cleaning Products Stabilized Against Microbial Attack

(71) Applicant: Thor GmbH, Speyer (DE)

(72) Inventors: Thomas Wunder, Neustadt a.d. Weinstrasse (DE); Roman Grabbe, Neustadt a.d. Weinstrasse (DE)

(73) Assignee: Thor GmbH, Speyer (DE)

(21) Appl. No.: 19/159,297

(22) PCT Filed: Feb. 16, 2024
(86) PCT No.: PCT/EP2024/000012

§ 371 Date: Aug. 22, 2025

(30) Foreign Application Priority Data
Feb. 23, 2023 (EP) 23000033.3

(51) Int. Cl. A61K 8/43; A61Q 5/02; A61Q 19/00; A61Q 19/10

(52) U.S. Cl. CPC A61K 8/43; A61Q 5/02; A61Q 19/007; A61Q 19/10

(57) ABSTRACT

The invention relates to the use of guanidine and/or at least one salt of guanidine for stabilizing aqueous products, such as cosmetic products and washing and cleaning products, against microbial attack.

The invention relates to the use of guanidine and/or at least one salt of guanidine for stabilization of aqueous products such as cosmetic products and washing and cleaning compositions against microbial infestation.

Water-containing products, such as washing and cleaning compositions or else cosmetic products, are typically produced using natural or biodegradable raw materials. These raw materials and the water used as solvent are frequently contaminated with microbes, such as bacteria, yeasts and fungi. If these products are produced without a preservative, they may already have high microbial counts one day after production.

In order to ensure that hygiene requirements are met by these products, and if appropriate also the constituents thereof used in production, and hence that the shelf life of the industrial products is assured, what are called biocides are usually added to the products, or to the constituents thereof.

One class of biocides used for preservation of aqueous products such as washing & cleaning compositions or else cosmetic products is that of the 3-isothiazolin-3-ones. This substance class includes antimicrobially very active compounds with a profile of action that differs to some degree. It is often also the case that synergistic combinations of different 3-isothiazolin-3-ones are used in order to reduce the amount of biocides used. For example, WO 99/08530 A1 suggests the combined use of methylisothiazolin-3-one and 1,2-benzisothiazolin-3-one.

A further class of biocides is that of what are called the alkylguanidinium salts and also polyguanidinium salts. For example, it is known from the “Directory of microbicides for the protection of materials”, Springer 2005, pages 726 to 731, that an antimicrobial effect is possessed by medium- and long-chain guanidines and biguanidines, such as cocopropylenediamine-1,5-bisguanidinium acetates, bis(guanidinooctyl)amine triacetates, poly(hexamethylenebiguanidine) hydrochloride, di(4′-chlorophenyldiguanido) hexane, chlorhexidine digluconate, chlorhexidine diacetate.

In addition to the use as a preservative, as disclosed in EP 1 646 429 A1, alkylguanidine compounds are used in cosmetics, especially in haircare.

In spite of the numerous known active ingredients and combinations of active ingredients or measures taken in the meantime, increasing regulatory restrictions in particular are causing an increased demand for novel preservation methods for cosmetic products and washing and cleaning compositions that preferably exclude or need only a reduced content of conventional preservatives or biocides.

The invention achieves this object through the novel use of guanidine and/or at least one salt of guanidine for stabilization of aqueous products selected from cosmetic products and washing and cleaning compositions against microbial infestation.

In the context of the invention, it has been found that, surprisingly, aqueous products, i.e. water-based products, as a result of the content of guanidine and/or of a salt of guanidine, which were unknown to date as antimicrobial actives, require significantly less and in some cases even none at all of the preservatives as typically present in these products for preservation. Nevertheless, these products are characterized by excellent stability to microbial infestation, especially during storage. Compared to products that do not contain guanidine or a salt of guanidine, the products stabilized against microbial infestation have significantly improved stability to infestation by microorganisms such as bacteria, yeasts and fungi, and are therefore less prone to microbial colonization. Furthermore, the cosmetic products, or the washing and cleaning compositions, are significantly improved with regard to their environmental compatibility, and also have all known properties in terms of good and safe use properties.

The guanidine is the compound of the formula shown below:

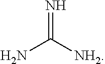

The salts of guanidine or guanidine salts in the context of the present invention are unsubstituted guanidine or guanidinium salts of the general formula shown below:

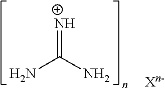

where n is 1, 2 or 3, and where the counterion Xⁿ⁻ is, for example, chloride, carbonate, nitrate, sulfate, thiocyanate, phosphate, hydrogencarbonate, formate, acetate or citrate.

In a preferred embodiment of the invention, the salt of guanidine used is guanidine hydrochloride and/or guanidine carbonate.

What is meant in the context of the invention by “at least one salt of guanidine” is that one or also two, three, four or more, i.e. a mixture of, salts of guanidine, are used in the aqueous products, or are present in these products.

The amount of guanidine, or the salt thereof, used for stabilization against microbial infestation may vary over a wide range and is generally in the range from 0.033% to 2.0% by weight, preferably in the range from 0.066% to 1.5% by weight, more preferably in the range from 0.1% to 1.0% by weight, based in each case on the guanidine content in the overall product. Any counterions other than H are therefore not taken into account in the determination of the guanidine content.

In the context of the present invention, the unit “% by weight” relates in each case to the entire aqueous product.

The pH of the aqueous products in which the guanidine and/or its salt(s) are used for stabilization against microbial infestation may vary over a wide range and is generally within a range from pH 6.5 to pH 11.5, preferably in the range from pH 7.0 to pH 11.0, more preferably in the range from pH 7.5 to pH 10.5. The pH can be adjusted to the claimed range with the aid of measures familiar to the person skilled in the art.

The water content of the aqueous products in which the guanidine and/or its salt(s) are used for stabilization against microbial infestation may vary over a wide range. The product is typically characterized in that it has a water content of greater than 1% by weight, preferably of greater than 5% by weight, more preferably of greater than 10% by weight, based on the overall product.

The present invention relates to the use of guanidine and/or at least one salt of guanidine for stabilization of aqueous products such as cosmetic products and washing and cleaning compositions against microbial infestation.

Useful cosmetic products are products from the field of cosmetics. These are, for example, products selected from the group consisting of shower gels, shower washes, dental cleaning and care products, mouthwashes, hair shampoos, hair conditioners, conditioning shampoos, hairsprays, hair conditioners, hair treatments, hair wraps, hair tonics, self-tanning agents, facial tonics, permanent wave fixing solutions, hair-dyeing shampoos, hair dyes, hair-setting agents, hair conditioners, hairstyling preparations, blow-waving lotions, hair-setting foams, hair gels, hair waxes, shaving foam, perfume preparations, wet wipes, care products and dermatological products for topical use, such as body creams, body lotions, face creams, sunscreen preparations and deodorants.

Useful washing and cleaning compositions include products from the field of washing and cleaning compositions that are selected from the group consisting of domestic cleaners, washing, cleaning or pretreatment compositions, refreshing sprays for textiles, fabric softeners, window cleaners, bathroom cleaners, toilet cleaners and dishwashing detergents.

As described above, the present invention relates to the novel use of guanidine and/or at least one salt of guanidine for stabilization of aqueous products, such as cosmetic products and washing and cleaning compositions, against microbial infestation. This novel use of the aforementioned compound that was not known to date as an antimicrobial active makes it possible to use significantly less, and in some cases even none at all, of the preservatives customarily used. The products stabilized against microbial infestation by the guanidine content nevertheless feature excellent resistance to microbial infestation, especially during storage.

In one embodiment of the invention, the product in which the guanidine or salt thereof is used for stabilization against microbial infestation is characterized in that the product contains one or more ingredient(s) having antimicrobial action as additional preservatives, selected from the group consisting of 5-chloro-2-methyl-4-isothiazolin-3-one, 2-methyl-4-isothiazolin-3-one, 1,2-benzisothiazolin-3-one, N-butyl-1,2-benzisothiazolin-3-one, N-methyl-1,2-benzisothiazolin-3-one, octylisothiazolinone, 2,2′-dithiobis [N-methylbenzamide], benzalkonium chloride, didecyldimethylammonium chloride, cetrimonium bromide, benzethonium chloride, polyhexamethylenebiguanide, bronopol, bronidox, chlorphenesin, dibromonitrilopropionamide, dibromodicyanobutane, iodopropynyl butylcarbamate, silver chloride, ethylhexylglycerol, hexylglycerol, pentane-1,2-diol, hexane-1,2-diol, heptane-1,2-diol, octane-1,2-diol, decane-1,2-diol, glyceryl caprylate, glyceryl caprate, glyceryl laurate, parabens, phenoxyethanol, phenylpropanol, phenethyl alcohol, benzyl alcohol, phenoxyisopropanol, isopropanol, ethylenediaminetetraacetic acid, sodium pyrithione, zinc pyrithione, piroctone olamine, o-phenylphenol, sorbic acid, formic acid, benzoic acid, propionic acid, salicylic acid, lactic acid, undecylenoic acid, dehydroacetic acid, 4-hydroxyacetophenone, o-cymen-5-ol, phenethyl alcohol, glyceryl caprylate, propanediol caprylate, climbazole, 4-(3-ethoxy-4-hydroxyphenyl)-2-butanone, anisic acid, levulinic acid, parabens and citric acid. The use amount of these additional preservatives may vary over a wide range. In a preferred embodiment of the invention, this/these are present in a total amount of less than 15 000 ppm, preferably less than 10 000 ppm, more preferably less than 5000 ppm, based on the overall product.

In a further embodiment of the invention, the product in which the guanidine or its salt is used for stabilization against microbial infestation is essentially free, preferably entirely free, of preservatives that are customarily used for preservation of cosmetic products and washing and cleaning compositions. In this embodiment of the invention, the aqueous product is characterized in that it contains one or more ingredient(s) selected from the group consisting of 5-chloro-2-methyl-4-isothiazolin-3-one, 2-methyl-4-isothiazolin-3-one, 1,2-benzisothiazolin-3-one, N-butyl-1,2-benzisothiazolin-3-one, N-methyl-1,2-benzisothiazolin-3-one, octylisothiazolinone, 2,2′-dithiobis [N-methylbenzamide], benzalkonium chloride, didecyldimethylammonium chloride, cetrimonium bromide, benzethonium chloride, polyhexamethylenebiguanide, bronopol, bronidox, chlorphenesin, dibromonitrilopropionamide, dibromodicyanobutane, iodopropynyl butylcarbamate, silver chloride, ethylhexylglycerol, hexylglycerol, pentane-1,2-diol, hexane-1,2-diol, heptane-1,2-diol, octane-1,2-diol, decane-1,2-diol, glyceryl caprylate, glyceryl caprate, glyceryl laurate, parabens, phenoxyethanol, phenylpropanol, phenethyl alcohol, benzyl alcohol, phenoxyisopropanol, isopropanol, ethylenediaminetetraacetic acid, sodium pyrithione, zinc pyrithione, piroctone olamine, o-phenylphenol, sorbic acid, formic acid, benzoic acid, propionic acid, salicylic acid, lactic acid, undecylenoic acid, dehydroacetic acid, 4-hydroxyacetophenone, o-cymen-5-ol, phenethyl alcohol, glyceryl caprylate, propanediol caprylate, climbazole, 4-(3-ethoxy-4-hydroxyphenyl)-2-butanone, anisic acid, levulinic acid, parabens and citric acid, in a total amount of less than 100 ppm, preferably less than 50 ppm, more preferably less than 10 ppm, based on the overall product.

The following examples serve for the further illustration of the present invention:

In order to demonstrate the stabilization of aqueous products against microbial infestation that are selected from washing and cleaning compositions and cosmetic products, a cleaning composition, a skin lotion and a shampoo were admixed with different amounts of guanidine salts and examined for their stability against microbial infestation:

The tested cleaning composition was an all-purpose cleaner without preservatives and having a pH of 7.7, the skin lotion was a body lotion without preservatives and having a pH of 7.1, and the shampoo was a hair shampoo without preservatives and having a pH of 6.9.

### Procedure:

The cleaning composition was admixed with particular concentrations of guanidine carbonate and guanidine hydrochloride and homogenized. The body lotion and the hair shampoo were each admixed and homogenized with guanidine hydrochloride and with octane-1,2-diol alone or in combination.

The batches of the products thus obtained were inoculated repeatedly at particular intervals with bacteria mixtures or yeast mixtures that are used as standard for microbiological stress tests of industrial products or cosmetic products (bacteria mixture 1 and 2, yeast mixture 1 and 2).

The cleaning composition was additionally inoculated repeatedly at particular intervals with a special inoculum consisting of a mixture of alkaliphilic bacteria (bacteria mixture 3) that had been isolated from a bacterially contaminated cleaning composition having a pH of 9.8.

Bacteria mixture 1:
Alcaligenes faecalis NCIMB 13145
Escherichia coli DSM 13631
Burkholderia cepacia DSM 9241
Staphylococcus aureus DSM 799
Pseudomonas aeruginosa DSM 50071
Pseudomonas putida DSM 13624
Pseudomonas spp. (Pseudomonas
oleovorans group) DSM 33933
Pseudomonas aeruginosa DSM 33935
Pseudomonas oleovorans DSM 33936
Pseudomonas putida DSM 33937
Pseudomonas aeruginosa DSM 33938
Bacteria mixture 2:
Pseudomonas aeruginosa ATCC 9027
Pseudomonas putida ATCC 31483
Burkholderia cepacia ATCC 25416
Klebsiella pneumoniae ATCC 10031
Pluralibacter gergoviae ATCC 33028
Escherichia coli ATCC 8739
Staphylococcus aureus ATCC 6538
Staphylococcus epidermidis ATCC 12228
Bacteria mixture 3:
Nesterenkonia spec. Thor 860
Nesterenkonia spec. Thor 861
Yeast mixture 1:
Pichia manshurica DSM 12249
Rhodotorula mucilaginosa DSM 12248
Candida boidinii DSM 13622
Candida albicans DSM 1386
Yeast mixture 2:
Candida albicans ATCC 10231
Candida parapsilosis ATCC 22019

The inoculum was added to the respective sample in each inoculation at 2% (v/w) as a fresh cell suspension in a physiological saline solution (0.9% NaCl). The cell count of the inoculum added was 10⁸-10⁹/ml for the bacteria mixtures, 10⁷-10⁸/ml for the yeast mixtures, consisting in equal parts of the individual bacteria strains or yeast strains.

The cell suspension of the bacteria mixtures was produced by growing the individual bacteria at 30° C. for 24 h in liquid culture on a shaker. The medium used to grow the bacteria from bacteria mixtures 1 and 2 was Müller-Hinton liquid medium. The bacteria from bacteria mixture 3 were grown in Müller-Hinton liquid medium that had been adjusted to pH 9.5 to 9.7 with an alkaline buffer solution beforehand. After growing, the individual liquid cultures were centrifuged, and the cells were resuspended in physiological saline solution. In order to adjust the mixtures to a total cell count of 10⁸ to 10⁹/ml, the individual bacteria suspensions, if necessary, were diluted accordingly before being combined with saline solution. The cell count of the inoculum suspensions was determined in each case by chamber counting.

The cell suspension of the yeast mixtures was produced by growing the yeast strains on Sabouraud glucose-agar at 25° C. for 48 hours and washing down the cell lawn in each case with physiological saline solution (0.9% NaCl). Further steps for establishment of a cell count of the inoculum suspension of 10⁷/ml to 10⁸/ml were effected analogously to the production of the bacteria suspensions.

In the examples, the samples were inoculated three times at weekly intervals with the respective cell suspensions (2% v/w) and the evolution of the bacteria or yeasts was followed by a semiquantitative evaluation method. After each inoculation, microbial infestation in the samples was evaluated in each case after incubation at 30° C. for 6 to 7 days. For this purpose, an aliquot was taken from each sample after homogenization using a sterile plastic inoculation loop (10 μl) and streaked evenly on agar plates (bacteria mixture 1 and 2 on tryptic soy agar, bacteria mixture 3 on DSMZ 31 alkaline nutrient agar, Sabouraud glucose-agar in the case of yeasts). The streaked agar plates were then incubated at 30° C. for 48 hours (bacteria mixture 1 and 2) or 5 to 7 days (bacteria mixture 3), or at 25° C. for 72 hours (yeast mixtures). After incubation, the bacteria colonies or yeast colonies that have grown on the agar plates were visually evaluated and quantified according to the following scheme:

| Evaluation | Growth on the agar surface |
|---|---|
| 0 | No growth |
| 1 | Minimal growth, 1 to 10 colonies visible over |
| | the entire streak |
| 2 | Light growth, maximum 100 colonies |
| 3 | Moderate growth, maximum 300 to 400 colonies |
| 4 | Uniform growth, >400 colonies, single colonies |
| | still clearly recognizable |
| 5 | Heavy growth, too many colonies to count, but |
| | no growth over the entire streak |
| 6 | Full growth, barely any single colonies still |
| | apparent, growth over the entire streak |

The results for the examples are shown in tables 1 to 5:

### EXAMPLE 1/TABLE 1

Inoculation 2% (w/v) with bacteria mixture 1, cell count 10⁹/ml

| | Bacterial growth after inoculation | | |
|---|---|---|---|
| | Incubation days [d] | | |
| | 1. | 2. | 3. |
| Test product/concentration | 6 d | 6 d | 6 d |
| All-purpose cleaner pH 7.7 | | | |
| No additives | 6 | 6 | 6 |
| No additives pH 9.5* | 4 | 6 | 6 |
| +1000 ppm guanidine carbonate | 0 | 0 | 5 |
| (656 ppm guanidine) pH 9.2 | | | |
| +1500 ppm guanidine carbonate | 0 | 0 | 0 |
| (984 ppm guanidine) pH 9.5 | | | |

*pH 9.5 established with NaOH

As can be inferred from the test results shown in table 1, the addition of 659 ppm of guanidine in the form of guanidine carbonate at a pH of pH 9.2 or pH 9.5 can achieve a distinct improvement in stability of the all-purpose cleaner to microbial infestation by bacteria.

### EXAMPLE 2/TABLE 2

Inoculation 2% (w/v) with bacteria mixture 3, cell count 10⁹/ml

| | Bacteria growth after inoculation | | |
|---|---|---|---|
| | Incubation days [d] | | |
| | 1. | 2. | 3. |
| Test product/concentration | 7 d | 7 d | 7 d |
| All-purpose cleaner pH 7.7 | | | |
| No additives | 6 | 6 | 6 |
| No additives pH 11.2* | 6 | 6 | 6 |
| +500 ppm guanidine carbonate | 0 | 2 | 2 |
| (328 ppm guanidine) pH 9.1 | | | |
| +1000 ppm guanidine carbonate | 0 | 0 | 1 |
| (656 ppm guanidine) pH 9.3 | | | |
| +2500 ppm guanidine carbonate | 0 | 0 | 0 |
| (1640 ppm guanidine) pH 10.0 | | | |
| +5000 ppm guanidine carbonate | 0 | 0 | 0 |
| (3280 ppm guanidine) pH 10.5 | | | |

*pH 11.2 established with NaOH

As can be inferred from the test results shown in table 2, even the addition of 330 ppm of guanidine in the form of guanidine carbonate at a pH of pH 9.1 can achieve a distinct improvement in stability of the all-purpose cleaner to microbial infestation by bacteria.

### EXAMPLE 3/TABLE 3

Inoculation 2% (w/v) with yeast mixture 1, cell count 10⁸/ml

| | Yeast growth after inoculation | | |
|---|---|---|---|
| | Incubation days [d] | | |
| | 1. | 2. | 3. |
| Test product/concentration | 6 d | 6 d | 6 d |
| All-purpose cleaner pH 7.7 | | | |
| No additives | 6 | 6 | 6 |
| No additives pH 10.5* | 3 | 4 | 6 |
| +1000 ppm guanidine carbonate | 1 | 1 | 3 |
| (656 ppm guanidine) pH 9.3 | | | |
| +2500 ppm guanidine carbonate | 0 | 0 | 0 |
| (1640 ppm guanidine) pH 9.9 | | | |
| +5000 ppm guanidine carbonate | 0 | 0 | 0 |
| (3280 ppm guanidine) pH 10.4 | | | |
| +1000 ppm guanidine hydrochloride | 5 | 6 | 6 |
| (618 ppm guanidine) pH 7.7 | | | |
| +2500 ppm guanidine hydrochloride | 1 | 1 | 1 |
| (1545 ppm guanidine) pH 7.7 | | | |
| +5000 ppm guanidine hydrochloride | 0 | 0 | 1 |
| (3090 ppm guanidine) pH 7.6 | | | |

*pH 10.5 established with NaOH

As can be inferred from the test results shown in table 3, even the addition of 659 ppm of guanidine in the form of guanidine carbonate at a pH of pH 9.9, or the addition of 1516 ppm of guanidine in the form of guanidine hydrochloride at a pH of 7.7, can achieve a distinct improvement in stability of the all-purpose cleaner to microbial infestation by yeasts.

### EXAMPLE 4/TABLE 4

Inoculation 2% (w/v) with bacteria mixture 3, cell count 10⁸/ml

| | Bacterial growth after inoculation | | |
|---|---|---|---|
| | Incubation days [d] | | |
| | 1. | 2. | 3. |
| Test product/concentration | 6 d | 6 d | 6 d |
| Body lotion pH 7.0 | | | |
| No additives | 6 | 6 | 6 |
| +10000 ppm guanidine hydrochloride | 6 | 6 | 6 |
| (6180 ppm guanidine) pH 6.8 | | | |
| +2500 ppm octanediol pH 7.0 | 4 | 6 | 6 |
| +5000 ppm octanediol pH 7.0 | 1 | 3 | 3 |
| +5000 ppm guanidine hydrochloride | 0 | 0 | 5 |
| (3090 ppm guanidine) + 2500 ppm | | | |
| octanediol pH 7.0 | | | |
| +7500 ppm guanidine hydrochloride | 0 | 0 | 0 |
| (4635 ppm guanidine) + 5000 ppm | | | |
| octanediol pH 6.9 | | | |

As can be inferred from the test results shown in table 4, even the addition of 3035 ppm of guanidine in the form of guanidine hydrochloride at a pH of pH 7.0 can achieve a distinct improvement in stability of the body lotion to microbial infestation by bacteria.

### EXAMPLE 5/TABLE 5

Inoculation 2% (w/v) with yeast mixture 2, cell count 10⁷/ml

| | Yeast growth after inoculation | | |
|---|---|---|---|
| | Incubation days [d] | | |
| | 1. | 2. | 3. |
| Test product/concentration | 6 d | 6 d | 6 d |
| Body lotion pH 7.0 | | | |
| No additives | 6 | 6 | 6 |
| +10000 ppm guanidine hydrochloride | 5 | 6 | 6 |
| (6180 ppm guanidine) pH 6.8 | | | |
| +2500 ppm octanediol pH 7.0 | 5 | 6 | 6 |
| +5000 ppm octanediol pH 7.0 | 3 | 3 | 2 |
| +5000 ppm guanidine hydrochloride | 2 | 3 | 3 |
| (3090 ppm guanidine) + 2500 ppm | | | |
| octanediol pH 7.0 | | | |
| +7500 ppm guanidine hydrochloride | 0 | 0 | 1 |
| (4635 ppm guanidine) + 5000 ppm | | | |
| octanediol pH 6.9 | | | |

As can be inferred from the test results shown in table 5, even the addition of 4553 ppm of guanidine in the form of guanidine hydrochloride at a pH of pH 6.9 can achieve a distinct improvement in stability of the body lotion to microbial infestation by yeasts.

### EXAMPLE 6/TABLE 6

Inoculation 2% (w/v) with yeast mixture 2, cell count 10⁷/ml

| | Yeast growth after inoculation | | |
|---|---|---|---|
| | Incubation days [d] | | |
| | 1. | 2. | 3. |
| Test product/concentration | 6 d | 6 d | 6 d |
| Shampoo pH 6.9 | | | |
| No additives | 5 | 6 | 6 |
| +10000 ppm guanidine hydrochloride | 4 | 6 | 6 |
| (6070 ppm guanidine) pH 6.8 | | | |
| +2500 ppm octanediol pH 6.9 | 4 | 5 | 6 |
| +5000 ppm octanediol pH 6.9 | 1 | 2 | 2 |
| +5000 ppm guanidine hydrochloride | 3 | 3 | 3 |
| (3090 ppm guanidine) + 2500 ppm | | | |
| octanediol pH 6.9 | | | |
| +7500 ppm guanidine hydrochloride | 0 | 0 | 0 |
| (4635 ppm guanidine) + 5000 ppm | | | |
| octanediol pH 6.8 | | | |

As can be inferred from the test results shown in table 6, even the addition of 4553 ppm of guanidine in the form of guanidine hydrochloride at a pH of pH 6.9 can achieve a distinct improvement in stability of the body lotion to microbial infestation by yeasts.

1. The use of guanidine and/or at least one salt of guanidine for stabilization of aqueous products selected from cosmetic products and washing and cleaning compositions against microbial infestation.

2. The use as claimed in claim 1, characterized in that the guanidine and/or its salt is/are used in an amount in the range from 0.033% to 2.0% by weight, based on the guanidine content in the overall product.

3. The use as claimed in claim 1, characterized in that the salt of guanidine used is guanidine hydrochloride and/or guanidine carbonate.

4. The use as claimed in claim 1, characterized in that the aqueous product has a pH in the range from pH 6.5 to pH 11.5.

5. The use as claimed in claim 1, characterized in that the aqueous product has a water content of greater than 1% by weight, preferably of greater than 5% by weight, more preferably of greater than 10% by weight, based on the overall product.

6. The use as claimed in claim 1, characterized in that the cosmetic product is selected from the group consisting of shower gels, shower washes, dental cleaning and care products, mouthwashes, hair shampoos, hair conditioners, conditioning shampoos, hairsprays, hair conditioners, hair treatments, hair wraps, hair tonics, self-tanning agents, facial tonics, permanent wave fixing solutions, hair-dyeing shampoos, hair dyes, hair-setting agents, hair conditioners, hairstyling preparations, blow-waving lotions, hair-setting foams, hair gels, hair waxes, shaving foam, perfume preparations, wet wipes, care products and dermatological products for topical use, such as body creams, lotions, face creams, sunscreen preparations or deodorants.

7. The use as claimed in claim 1, characterized in that the laundry detergent and cleaning composition is selected from the group consisting of domestic cleaners, washing, cleaning or pretreatment products, refreshing sprays for textiles, fabric softeners, window cleaners, bathroom cleaners, toilet cleaners and dishwashing detergents.

8. The use as claimed in claim 1, characterized in that the aqueous product is characterized in that it contains one or more ingredient(s) selected from the group consisting of 5-chloro-2-methyl-4-isothiazolin-3-one, 2-methyl-4-isothiazolin-3-one, 1,2-benzisothiazolin-3-one, N-butyl-1,2-benzisothiazolin-3-one, N-methyl-1,2-benzisothiazolin-3-one, octylisothiazolinone, 2,2′-dithiobis[N-methylbenzamide], benzalkonium chloride, didecyldimethylammonium chloride, cetrimonium bromide, benzethonium chloride, polyhexamethylenebiguanide, bronopol, bronidox, chlorphenesin, dibromonitrilopropionamide, dibromodicyanobutane, iodopropynyl butylcarbamate, silver chloride, ethylhexylglycerol, hexylglycerol, pentane-1,2-diol, hexane-1,2-diol, heptane-1,2-diol, octane-1,2-diol, decane-1,2-diol, glyceryl caprylate, glyceryl caprate, glyceryl laurate, parabens, phenoxyethanol, phenylpropanol, phenethyl alcohol, benzyl alcohol, phenoxyisopropanol, isopropanol, ethylenediaminetetraacetic acid, sodium pyrithione, zinc pyrithione, piroctone olamine, o-phenylphenol, sorbic acid, formic acid, benzoic acid, propionic acid, salicylic acid, lactic acid, undecylenoic acid, dehydroacetic acid, 4-hydroxyacetophenone, o-cymen-5-ol, phenethyl alcohol, glyceryl caprylate, propanediol caprylate, climbazole, 4-(3-ethoxy-4-hydroxyphenyl)-2-butanone, anisic acid, levulinic acid, parabens and citric acid, in a total amount of less than 100 ppm, based on the overall product.
